(19) United States
(12) Patent Application Publication
(10) Pub. No.: US 2017/0007390 A9
(43) Pub. Date: Jan. 12, 2017

(54) PERCUTANEOUS TRANSLUMINAL ANGIOPLASTY DEVICE WITH INTEGRAL EMBOLIC FILTER

(71) Applicant: Contego Medical, LLC, (US)

(72) Inventors: Ravish Sachar, Raleigh, NC (US); Udayan G. Patel, San Jose, CA (US)

(21) Appl. No.: 13/838,523

(22) Filed: Mar. 15, 2013

Related U.S. Application Data
(63) Continuation-in-part of application No. 11/763,118, filed on Jun. 14, 2007.
Application No. 11/763,118 is a continuation-in-part of application No. 10/997,803, filed on Nov. 24, 2004, now Pat. No. 8,403,976.
Application No. 10/997,803 is a continuation-in-part of application No. 12/604,236, filed on Oct. 22, 2009.
(60) Provisional application No. 61/107,391, filed on Oct. 22, 2008.
(60) Provisional application No. 61/107,395, filed on Oct. 22, 2008.
(60) Provisional application No. 61/107,404, filed on Oct. 22, 2008.
(60) Provisional application No. 60/813,395, filed on Jun. 14, 2006.

(51) Int. Cl. A61F 2/01

(52) U.S. Cl. CPC A61F 2/013

(57) ABSTRACT

A percutaneous transluminal angioplasty device includes an embolic filter mounted to the catheter shaft at a location distal to the angioplasty balloon. Thus the filter can be down-stream from the blockage and can be properly positioned to capture embolic particles that may be set loose into the blood stream as the angioplasty procedure can be performed. The embolic filter can be normally un-deployed against the catheter shaft to facilitate introduction and withdrawal of the device to and from the operative site. Once the angioplasty balloon can be properly positioned, however, means operatively associated with the embolic filter can be actuated to deploy the filter to position a filter mesh across the lumen of the vessel.

### CROSS-REFERENCE TO RELATED APPLICATION

This application is a continuation in part of U.S. patent application Ser. No. 11/763,118, filed Jun. 14, 2007, currently pending, which is a continuation-in-part of U.S. patent application Ser. No. 10/997,803, filed Nov. 24, 2004, currently pending. This application further claims priority to Provisional Patent Application No. 60/813,395, filed Jun. 14, 2006. This application also claims priority to U.S. patent application Ser. No. 12/604, 236, filed on Oct. 22, 2009, currently pending, which claims priority to U.S. provisional application Ser. No. 61/107,391 filed on Oct. 22, 2008, U.S. provisional application Ser. No. 61/107,395 filed on Oct. 22, 2008 and U.S. provisional application Ser. No. 61/107,404 filed on Oct. 22, 2008.

### BACKGROUND

1. Field of the Invention

Implementations described herein relate generally to surgical devices and relate more specifically to percutaneous transluminal angioplasty devices.

2. Related Art

The vascular bed supplies a constant flow of oxygen-rich blood to the organs. In diseased vessels, blockages can develop that can reduce blood flow to the organs and cause adverse clinical symptoms up to and including fatality. Diseased vessels can comprise a range of material from early-stage thrombosis to late-stage calcified plaque.

Angioplasty can be described as a catheter-based procedure performed by a physician to open up a blocked vessel and restore blood flow. An entry site can be opened, for example, in the patient's groin, arm, or hand, and a guide wire and catheter can be advanced under fluoroscopic guidance to the location of the blockage. A catheter having a small balloon adjacent its distal end can be advanced under fluoroscopic guidance until the balloon lies within the stenosed region. The balloon can be then inflated and deflated one or more times to expand the stenosed region of the artery.

Angioplasty can release embolic particles down-stream from the stenosed location. These embolic particles can result in adverse clinical consequences. It has been shown beneficial to trap these embolic particles to prevent them from traveling downstream with blood flow to the capillary bed (e.g., Baim D S, Wahr D, George B, et al., Randomized trial of a distal embolic protection device during percutaneous intervention of saphenous vein aorto-coronary bypass grafts, Circulation 2002; 105:1285-90).

In addition to balloon angioplasty, stenoses can also be treated with stents and with mechanical atherectomy and thrombectomy devices. These devices can be also prone to releasing embolic particles downstream from the stenosed location.

Systems available today used to catch these embolic particles consist primarily of filter systems or occlusion balloon systems, both built on a guidewire. Typically, a filter scaffolding configured to support a filter membrane is mounted at the distal end of the filter guidewire. The filter scaffolding is movable between a retracted position, in which the scaffolding lies against the guidewire for insertion and retraction of the guidewire in the patient's body, and an expanded position in which the filter medium expands across substantially the entire vessel. In use, the prior art filter guidewire is inserted through the main lumen of the angioplasty catheter and advanced to a “landing zone” distal to the stenosis. The filter guidewire is then manipulated to deploy a filter scaffolding having a filter medium attached and configured to capture any emboli released by the angioplasty procedure.

These systems suffer shortcomings related to simplicity of use and crossing tight lesions with a filter or balloon guidewire that can be larger in diameter than the guidewire which would normally be used. These embolic protection guidewires also suffer from flexibility and stability problems that render the protected angioplasty procedure relatively more difficult in many cases. In the case of saphenous vein grafts, the problems relate specifically to aorto-ostial lesions, where the guidewire may not be long enough to provide support, or distal vein graft lesions and renal artery lesions, where there can be not enough of a landing zone for the filter. The latter can be a problem as currently available filter systems can have a considerable distance between the treatment balloon and the distal filter. This distance can be a problem not only in distal vein graft lesions, but also in arterial stenoses in which there can be a side branch immediately after the stenosis, such as native coronary arteries. In such cases, the filter can often be deployed only distal to the side branch, thus leaving the side branch unprotected from embolic particles.

Accordingly, a need exists for improved percutaneous transluminal angioplasty devices having an integral embolic filter.

### SUMMARY

It is to be understood that this summary is not an extensive overview of the disclosure. This summary is exemplary and not restrictive, and it is intended to neither identify key or critical elements of the disclosure nor delineate the scope thereof. The sole purpose of this summary is to explain and exemplify certain concepts of the disclosure as an introduction to the following complete and extensive detailed description.

Stated generally, the present disclosure comprises a percutaneous transluminal angioplasty device with integral embolic filter. Because the filter can be integral with the catheter of the angioplasty device, any need to insert a separate device into the vessel can be eliminated. Further, proper placement of the angioplasty balloon can assure proper placement of the embolic filter.

Stated more specifically, the present disclosure comprises a catheter having an elongated shaft, proximal and distal ends, a longitudinal axis and a filter. The filter comprises a first ring coaxially fixedly mounted on a distal portion of the catheter shaft, a second ring coaxially slidably mounted on a distal portion of the catheter shaft and configured to be moved toward and away from the first ring and a scaffolding extending between the first and second rings. The scaffolding further comprises a plurality of first longitudinal connecting members, each having a first end attached to the first ring and a second end extending toward the second ring; a plurality of second longitudinal connecting members, each having a first end attached to the second ring and a second end extending toward the first ring. Each of the first and second longitudinal connecting members further comprise a bifurcation formed on the second end thereof, each of the bifurcations comprising first and second branches; and a means for connecting a branch on each of the plurality of first longitudinal connecting members to a branch on an opposite one of the plurality of second longitudinal connecting members. The filter further comprises a membrane connected to at least the scaffolding.

Additional features and advantages of exemplary implementations of the disclosure will be set forth in the description which follows, and in part will be obvious from the description, or may be learned by the practice of such exemplary implementations. The features and advantages of such implementations may be realized and obtained by means of the instruments and combinations particularly pointed out in the appended claims. These and other features will become more fully apparent from the following description and appended claims, or may be learned by the practice of such exemplary implementations as set forth hereinafter.

### BRIEF DESCRIPTION OF THE DRAWINGS

The accompanying drawings, which are incorporated in and constitute a part of this specification, illustrate aspects and together with the description, serve to explain the principles of the methods and systems.

FIG. 1 illustrates a side view of one aspect of an angioplasty device with integral embolic filter.

FIG. 2A illustrates a cross-section of the proximal end of the angioplasty device with integral embolic filter shown in FIG. 1; and FIG. 2B illustrates a cross-section of the distal end of the device shown in FIG. 1.

FIG. 3 illustrates a schematic view of one aspect of a filter scaffolding of the angioplasty device of FIG. 1, showing the filter scaffolding in an un-deployed position.

FIG. 4 illustrates a schematic view of the filter scaffolding of FIG. 3, showing the filter scaffolding in a deployed position.

FIG. 5 illustrates a schematic view of another aspect of a filter scaffolding of the angioplasty device of FIG. 1, showing the filter scaffolding in an un-deployed position.

FIG. 6 illustrates a schematic view of the filter scaffolding of FIG. 5, showing the filter scaffolding in a deployed position.

FIG. 7 illustrates a schematic view of a third aspect of a filter scaffolding of the angioplasty device of FIG. 1, showing the filter scaffolding in an un-deployed position.

FIG. 8 illustrates a schematic view of the filter scaffolding of FIG. 7, showing the filter scaffolding in a deployed position.

FIG. 9 illustrates a blood vessel having a stenosis.

FIG. 10 illustrates the blood vessel with stenosis of FIG. 9 with the angioplasty device of FIG. 1 positioned therein.

FIG. 11 illustrates the blood vessel and angioplasty device of FIG. 10 with the integral embolic filter expanded.

FIG. 12 illustrates the blood vessel and angioplasty device of FIG. 10 with the angioplasty balloon and integral embolic filter deployed.

FIG. 13 illustrates the blood vessel and angioplasty device of FIG. 10 after treatment of the stenosis, with the angioplasty balloon in its un-deployed position and the embolic filter still in its deployed position.

FIG. 14 illustrates the blood vessel and angioplasty device of FIG. 10 after treatment of the stenosis, with both the angioplasty balloon and embolic filter in an un-deployed position in preparation for withdrawal of the device from the vessel.

FIG. 15 illustrates an alternate aspect of a filter scaffolding comprising a sinusoidal frame.

FIG. 16 illustrates one aspect of the attachment of the sinusoidal frame.

FIG. 17 illustrates a side view of another aspect of an angioplasty device with integral embolic filter where the treatment device lies distal to the filter.

### DETAILED DESCRIPTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention provided as an enabling teaching of the invention in its best, currently known aspect. To this end, those skilled in the relevant art will recognize and appreciate that many changes can be made to the various aspects of the invention described herein, while still obtaining the beneficial results described herein. It will also be apparent that some of the desired benefits described herein can be obtained by selecting some of the features described herein without utilizing other features. Accordingly, those who work in the art will recognize that many modifications and adaptations to the present invention are possible and can even be desirable in certain circumstances and are a part described herein. Thus, the following description is provided as illustrative of the principles described herein and not in limitation thereof.

Reference will be made to the drawings to describe various aspects of one or more implementations of the invention. It is to be understood that the drawings are diagrammatic and schematic representations of one or more implementations, and are not limiting of the present disclosure. Moreover, while various drawings are provided at a scale that is considered functional for one or more implementations, the drawings are not necessarily drawn to scale for all contemplated implementations. The drawings thus represent an exemplary scale, but no inference should be drawn from the drawings as to any required scale.

In the following description, numerous specific details are set forth in order to provide a thorough understanding described herein. It will be obvious, however, to one skilled in the art that the present disclosure may be practiced without these specific details. In other instances, well-known aspects of percutaneous transluminal angioplasty devices and embolic filters have not been described in particular detail in order to avoid unnecessarily obscuring aspects of the disclosed implementations.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

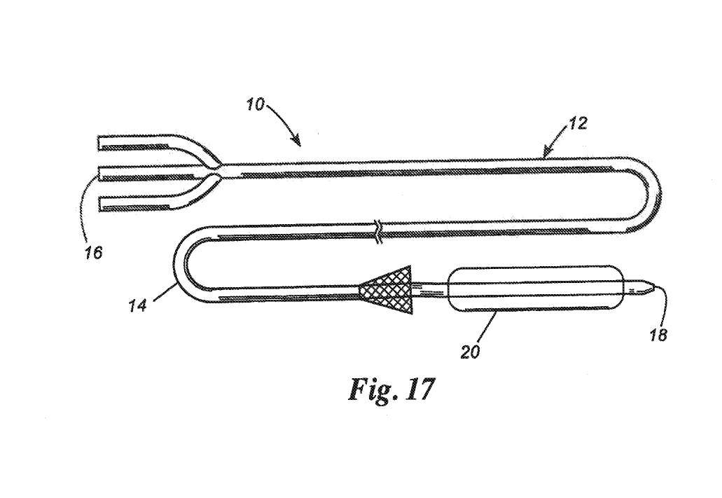

Referring now to the drawings, in which identical numbers indicate identical elements throughout the various views, FIG. 1 illustrates a first aspect of an angioplasty catheter with integral embolic filter 10 according to the present invention. The angioplasty catheter with integral embolic filter 10 comprises an elongated catheter 12 having a shaft 14 with a proximal end 16 and a distal end 18. As used herein, “proximal” refers to the portion of the device closest to the physican performing the procedure and “distal” refers to the portion of the device that is furthest from the physician performing the procedure. An angioplasty treatment device 20 can be mounted to the catheter 12 at a location near the distal end 18 of the catheter shaft 14. Angioplasty treatment devices comprise, for example and without limitation, inflatable balloons, expandable stents, atherectomy and thrombectomy devices and the like. An embolic filter 30 can be mounted to the catheter shaft 14 at a location distal to the angioplasty treatment device 20 and at or proximal to the distal end 18 of the catheter 12. As illustrated in FIG. 17, it is also contemplated that the embolic filter 30 can be mounted to the catheter shaft 14 at a location proximal to the treatment device 20. In additional or alternative embodiments, the filter 30 can be oriented to face towards or away from the treatment device. One skilled in the art will also appreciate in light of the present disclosure that the angioplasty catheter can be configured to be, for example and without limitation, an over-the-wire catheter, a rapid-exchange catheter and the like. It is solely for clarity of disclosure that the present description describes an over-the-wire catheter modality.

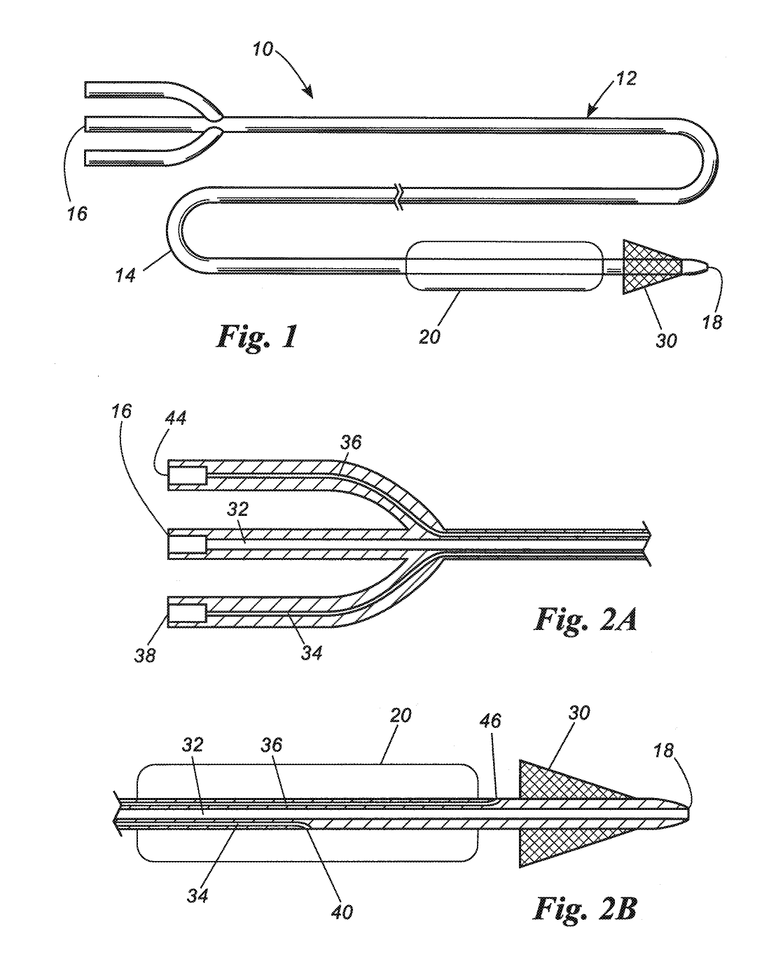

Referring now to FIG. 2, the catheter shaft 14 can define three lumens: a main lumen 32, an angioplasty balloon inflation lumen 34, and an embolic filter actuator wire lumen 36. The main lumen 32 can extend from the proximal end 16 to the distal end 18 of the catheter shaft 14. The main lumen 32 can optionally provide a working channel and be configured to receive a guidewire therethrough for advancing the distal end 18 of the catheter 12 through the patient's vasculature to a treatment site. As used herein, the term “treatment site” refers to the location of the occlusion within the patient's vasculature, and when the catheter 12 is referred to as being located or positioned at the treatment site, it will be understood to mean that the catheter is positioned such that the angioplasty treatment device 20 is located within the occlusion.

The balloon inflation lumen 34 can extend from a proximal port 38 at the proximal end 16 of the catheter 12 and through the catheter shaft 14 to a distal port 40 located within the angioplasty treatment device 20. Similarly, the actuator wire lumen 36 can extend from a proximal port 44 at the proximal end 16 of the catheter 12 and through the catheter shaft 14 to a distal port 46 distal to the angioplasty treatment device 20.

Unless otherwise stated, all of the aspects disclosed below share the foregoing characteristics, and the various aspects differ primarily in the design of the embolic filter. Thus, as the various aspects are disclosed, it will be understood unless stated otherwise that each aspect includes the foregoing features, and the description will instead focus on the design and operation of the embolic filter.

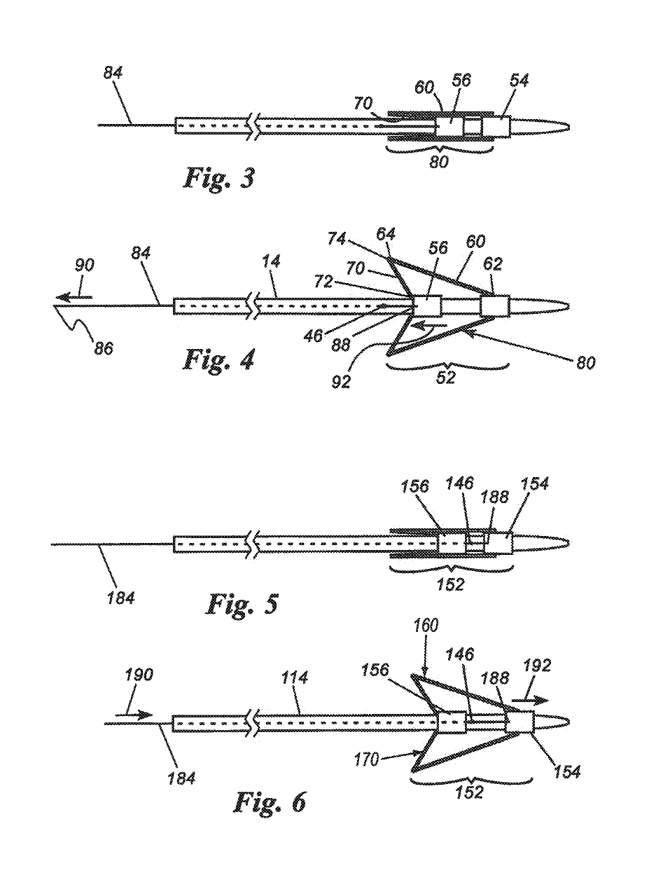

Referring to aspects of the present disclosure illustrated in FIGS. 3 and 4, the embolic filter 30 comprises a filter membrane 50 (FIG. 12) having holes selectively sized to permit the passage of blood but to capture particles larger than normal blood particles and a collapsible scaffolding 52 for supporting the filter membrane. For clarity of illustration, the drawing figures omit the filter membrane 50 when illustrating the scaffolding 52, but it will be understood that all embolic filters disclosed in this application comprise a filter membrane supported by the scaffolding. It is contemplated that the scaffolding 52 can include a proximal ring 56 and a distal ring 54. In one aspect, both of the rings can be located between the distal end of the angioplasty treatment device 20 and the distal end 18 of the catheter shaft. In a further aspect, the distal ring 54 can be fixed in place on the catheter shaft 14, and the proximal ring 56 can be slidably mounted to the catheter shaft for axial movement in the proximal and distal directions.

Each of a plurality of first strut sections 60 can have a first end 62 and a second end 64. The first end 62 of each first strut section 60 can be attached to the distal ring 54, and each first strut section can extend in the proximal direction.

In other aspects, each of a corresponding plurality of second strut sections 70 can have a first end 72 and a second end 74. Here, the first end 72 of each second strut section 70 can be attached to the proximal ring 56, and each second strut section can also extend in the proximal direction.

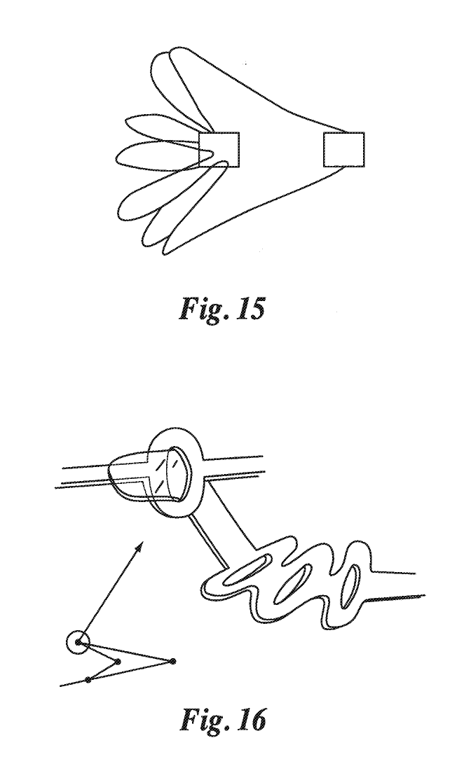

In yet other aspects, the plurality of second strut sections 70 can be replaced with a sinusoidal ring structure 55 as illustrated in FIGS. 15-16. In this aspect, the sinusoidal ring 55 contracts radially inward as the relative distance between the distal and proximal rings increases and expands as the relative distance between the distal and proximal rings decreases.

In yet other aspects, the second end 64 of each first strut section 60 can attach to the second end 74 of a corresponding second strut section 70. Here, each connected first and second strut section 60, 70 collectively comprises a strut 80. As one skilled in the art will appreciate from the discussion supra, a plurality of strut 80 can be spaced circumferentially about and connecting the proximal and distal rings to form the scaffolding 52. In operation and as shown in FIG. 3, when the proximal and distal rings 56, 54 are adjacent one another each strut 80 can be configured to fold back upon itself. Additionally, when the proximal ring 56 is proximally displaced from the distal ring 54, the struts 80 can be configured to open in a manner similar to an umbrella. The filter membrane 50 can be supported on the first strut sections 60 such that when the scaffolding 52 opens, as shown in FIG. 4, the filter membrane can deploy in a manner similar to an umbrella canopy.

It is contemplated that each strut can further comprise at least one “zone of weakness,” i.e., a zone of the strut that can be configured to be physically weaker than the majority of the strut in order to control the locations at which the struts bend. One skilled in the art will appreciate that the at least one zone of weakness can be formed in any of a number of ways. In one aspect, a notch can be formed in one or both sides of the strut. In another aspect, at least one of the upper surface and lower surface of the strut can be scored. In another aspect, the at least one zone of weakness can be formed of a material that can be structurally weaker than the material comprising the remainder of the strut. In yet other aspects, the at least one zone of weakness can comprise mechanical hinges. In yet other aspects and as shown in FIG. 15, the apices of the sinusoidal ring 55 comprise a zone of weakness. In even further aspects, at least two of these approaches can be combined to form the at least one zone of weakness, e.g., both notching the width and scoring the depth of the strut. In addition, the at least one zone of weakness can comprise a plurality of one type of physical arrangement, e.g., a single zone of weakness can comprise a plurality of notches or a plurality of scores. In operation, the at least one zone of weakness can be configured to bend the strut in response to a force at a predetermined angle to the longitudinal axis of that portion of the strut.

In operation, movement of the proximal ring 56 toward and away from the distal ring 54 to open and to close the embolic filter 30 can be accomplished by manipulation of an actuator wire 84. In one aspect, the proximal end 86 of the actuator wire 84 can extend out of the proximal port 44 of the actuator wire lumen 36 so as to be controllable by the physician performing the procedure. Here, the actuator wire 84 can extend through the actuator wire lumen 36 and can exit through the distal port 46 of the actuator wire lumen. In another aspect, the distal end 88 of the actuator wire 84 can be attached to the proximal ring 56.

One skilled in the art will appreciate here are a variety of ways in which the filter scaffolding 52 and actuator wire 84 can be arranged to permit the embolic filter 30 to be opened and closed by moving the proximal end 86 of the actuator wire. In a first aspect, the filter scaffolding 52 can be formed in a normally closed or undeployed position. In operation, pulling the proximal end 86 of the actuator wire 84 can cause the proximal ring 56 to slide in a proximal direction to open the filter scaffolding 52. The filter scaffolding can be configured so that releasing the tension on the actuator wire 84 and/or pushing the actuator wire 84 distally can permit the filter scaffolding 52 to collapse to an un-deployed position.

In another aspect of the present disclosure illustrated in FIGS. 5 and 6, a filter scaffolding 152 can comprise a proximal ring 156 that can be fixed with respect to a catheter shaft 114 and a distal ring 154 that can be slidably positioned along the catheter shaft in the proximal and distal directions. In a further aspect, a distal port 146 of an actuator wire lumen 136 can be located distal to the proximal ring 156. Here, an actuator wire (not shown) can extend through the actuator wire lumen, can exit through a distal port 146, and can attach to the distal ring 154. The filter scaffolding 152 can be formed in a normally closed position. In operation, pushing the actuator wire 184 can displace the distal ring 154 in a distal direction away from the proximal ring 156 to deploy the filter scaffolding 152. The filter scaffolding can be configured so that releasing the force on the actuator wire 184 and/or pushing the actuator wire 184 distally can permit the filter scaffolding 152 to return to its un-deployed position.

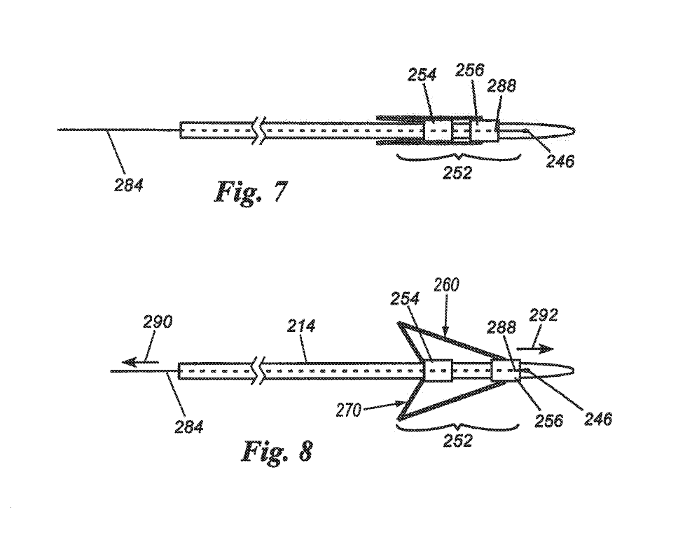

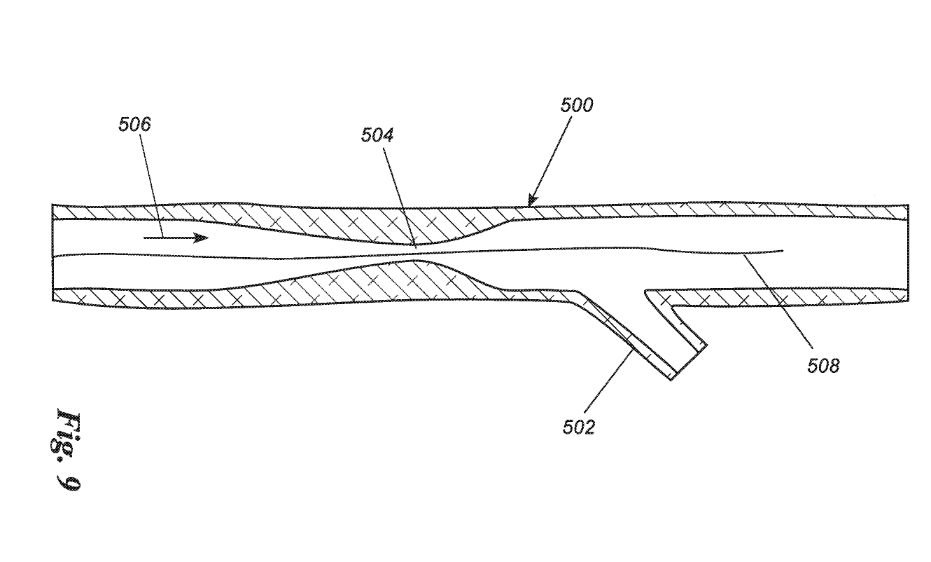

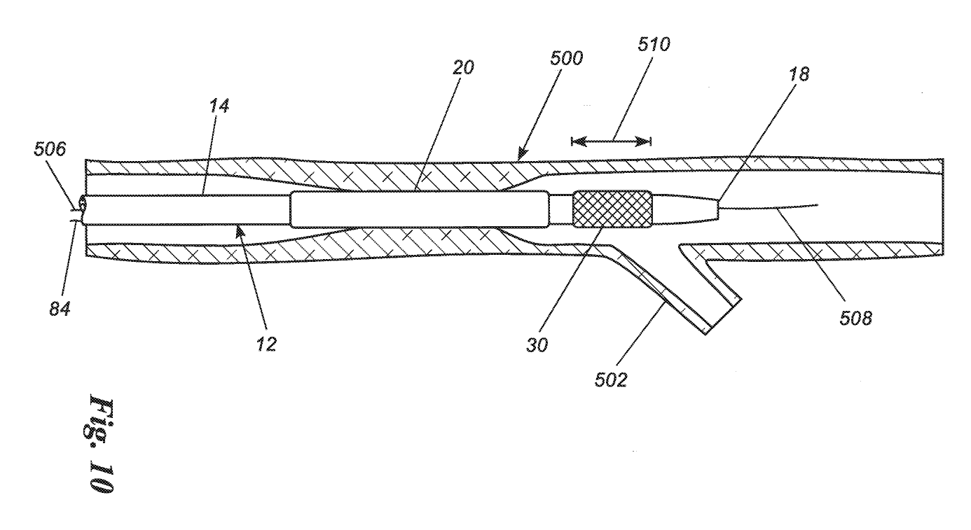

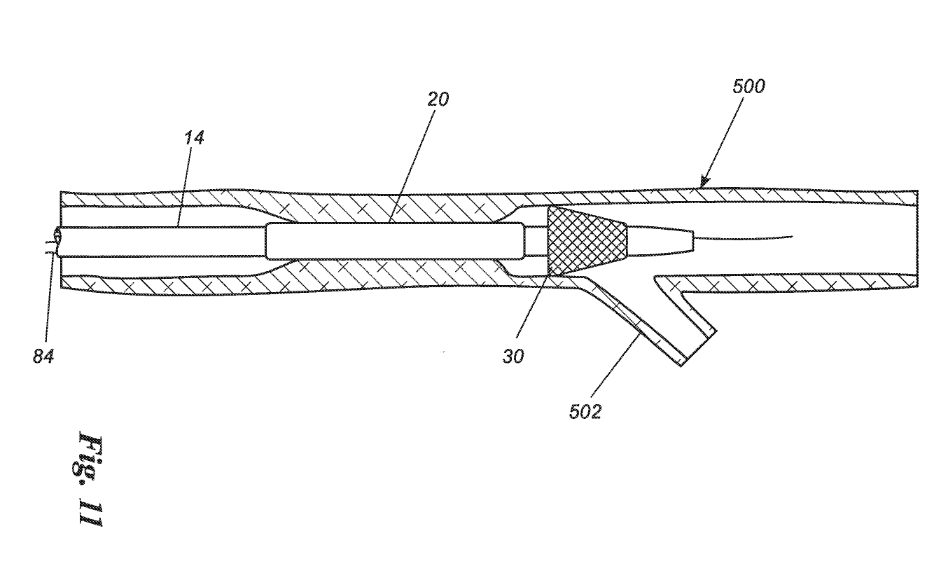

In yet another aspect of the present disclosure illustrated in FIGS. 7 and 8, a proximal ring 254 can be fixed with respect to a catheter shaft 214, and a distal ring 256 can be slidably positioned along the catheter shaft in the proximal and distal directions. In a further aspect, a distal port 246 of an actuator wire lumen 236 can be located distal to the distal ring 256. Here, an actuator wire 284 can extend through the actuator wire lumen 236, can exit through the distal port 246, and can attach to the distal ring 256. The filter scaffolding 252 can be formed in a normally closed position. In operation, pulling on the actuator wire 284 can displace the distal ring 256 in a distal direction and away from the proximal ring 156 to deploy the filter scaffolding 252. The filter scaffolding can be configured so that releasing the force on the actuator wire 284 can permit the filter scaffolding 252 to return to its un-deployed position.

Referring back to FIGS. 3 and 4, another aspect of a filter scaffolding can be structurally identical to the first embodiment 52 except that the filter scaffolding can be formed in a normally open or deployed position. Here, it is contemplated that application of a distally directed force to the proximal end 86 of the actuator wire 84 (i.e., pushing the actuator wire) can maintain the proximal ring 56 in its distal position and hence can maintain the filter scaffolding 52 in its un-deployed position. The filter scaffolding 52 can be permitted to expand to its normally deployed position, expanding the filter membrane 50, upon release of the force applied to the actuator wire 84. Immediately after completion of the interventional procedure, a distally directed force can again be applied to the proximal end 86 of the actuator wire 84, moving the proximal ring 56 toward the distal ring 54 and collapsing the filter scaffolding 52.

Referring back to FIGS. 5 and 6, a fifth aspect can be structurally identical to the third aspect with the exception that the filter scaffolding 152 can be formed in a normally open position. Here, it is contemplated that the distal ring 154 can be normally displaced toward the distal end 18 of the catheter shaft 114. In operation, pulling on the distal end 188 of the actuator wire 184 can move the distal ring 154 proximally toward the fixed proximal ring 156, collapsing the filter scaffolding 152 while releasing the tension on the actuator wire 184 can permit the filter scaffolding 152 to expand to its deployed position.

In those aspects in which the force applied to the actuator wire is configured to be an axial compressive force, those skilled in the art can appreciate that a stiffer wire can be used to prevent buckling of the actuator wire than in those embodiments where the force applied to the actuator wire is configured to be an axial tensile force.

In the present disclosure, and especially in the case of actuator wires, the term “wire” is intended to comprise, for example and without limitation, metallic wires, polymeric wires, and the like. In the case of polymeric wires, the polymers used can comprise, for example and without limitation, nylon, polypropylene and the like.

In the foregoing aspects, the filter membrane 50 can be formed from at least one of a textile, a polymer and a wire mesh. In another aspect, the filter membrane 50 comprises pores and, in a further aspect, the pores can be sized to allow blood to pass but not embolic particles. It is also contemplated that the filter membrane 50 can be mounted either on top of or inside of the frame.

In the foregoing aspects, the filter membrane 50 can be configured to cover the exterior surface of the outermost strut sections, i.e., the first strut sections 60, 160, and 260. Optionally, the filter membrane 50 can be further configured to extend beyond the distal or second ends 64, 164, and 264 of the first strut sections 60, 160, and 260, where it can be attached to the circumference of the distal ring 54, 156, 256. In those aspects in which the distal ring 54 can be fixed, the filter membrane 50 can optionally be configured to extend beyond the distal end of the distal ring and can be attached to the circumference of the catheter shaft 14 at a location between the distal ring 54 and the distal end 18 of the catheter shaft.

It is also contemplated that the filter membrane 50 in each of the disclosed embodiments can be attached to the inner surfaces of the first strut sections 60, 160, and 260 instead of to the outer surfaces.

It is further contemplated that the inner or second strut sections 70, 170, 270 can also be configured in a concave shape with respect to the blood flow when the filter scaffolding is deployed. In further or additional aspects, the filter membrane 50 can be attached to the inner or outer surfaces of the second strut sections 70, 170, 270. When the filter membrane 50 is attached to the surfaces of the second strut sections 70, 170, 270, the filter membrane 50 can optionally extend beyond the distal or second ends 74, 174, 274 of the second strut sections and be attached to the circumference of the proximal ring 56, 154, 254. It is also contemplated that, if the filter membrane 50 can be attached to the outer surfaces of the second strut sections 70 and the proximal ring 56 can be fixed, the filter membrane can be configured to extend beyond the distal end of the proximal ring and can be attached to the catheter shaft 14 at a location between the proximal and distal rings 56, 54.

In all of the foregoing instances, the filter scaffolding comprises a fixed ring and a movable ring, raising the filter can be accomplished by moving the rings apart, and collapsing the filter can be achieved by moving the rings together. “Moving apart” and “moving together” are used as relative terms, such that only one of the two rings need move with respect to the other ring for the rings to “move apart” or “move together.”

Similarly, the process of raising and collapsing the filter can be thought of as being viewed from the perspective of the catheter, such that a movable ring can be moved toward or away from a fixed ring.

In all of the foregoing instances, one can appreciate that both actively applying a force to move a ring and releasing a force to permit the ring to move of its own accord comprise a step of “causing” the movable ring to move by “controlling” the actuator wire. Thus, in both the normally deployed and normally un-deployed filter scaffolding embodiments described herein, the actuator wire can be “controlled” to “cause” a movable ring to move, whether that control takes the form of applying or releasing a force on the actuator wire.

It is also contemplated that, rather than having the physician directly grasp the proximal end of the actuator wire, a control device can be associated with the proximal end of the actuator wire at the proximal end of the catheter shaft. The control device can incorporate, for example and without limitation, levers, sliders, rotating spindles, or the like to facilitate movement of the wire. One example of such a mechanical arrangement is described in U.S. Patent Publication No. US 2010/0106182, paragraphs [0079]-[0090] and FIGS. 29-33, which disclosure is hereby incorporated by reference.

Use of the angioplasty device with integral embolic filter described above to treat a stenosis in a blood vessel can be shown in FIGS. 9-13. In FIG. 9, a vessel 500 can have a branch vessel 502 diverging from it. The vessel 500 can have a stenosis 504. The direction of blood flow through the vessel 500 is indicated by the arrow 506. A guide wire 508 has been inserted by the physician as a preliminary step in the interventional procedure.

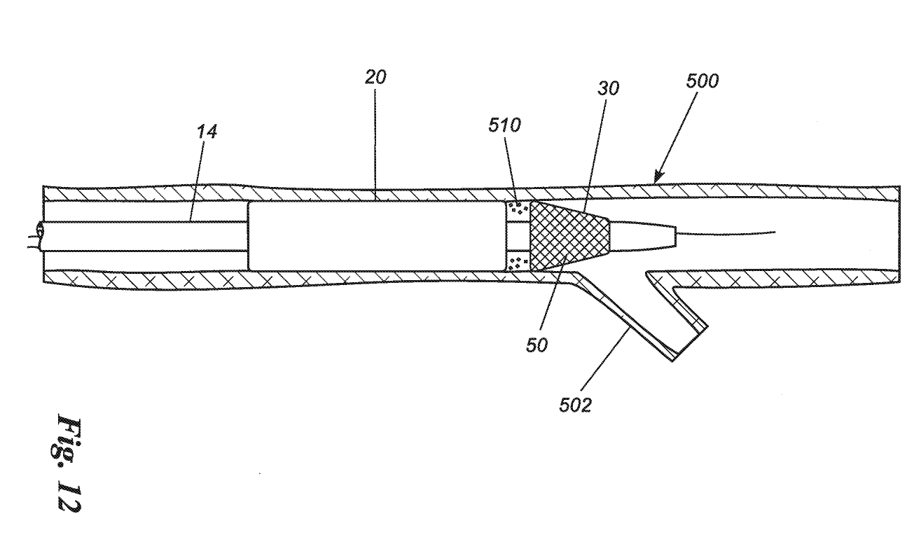

FIG. 10 shows the catheter 12 with angioplasty balloon 20 and embolic filter 30 in their un-deployed positions and lying adjacent to the catheter shaft 14. The distal end 18 of the catheter shaft 14 has been advanced over the guide wire 506 until the deflated angioplasty balloon 20 resides within the stenosis. With the catheter 12 positioned such that the angioplasty balloon 20 can be located within the stenosis, the catheter can be said to be at its “target site.” With the catheter at the target site, the portion of the vessel 500 occupied by the embolic filter 30 can be referred to as the “landing zone” 510.

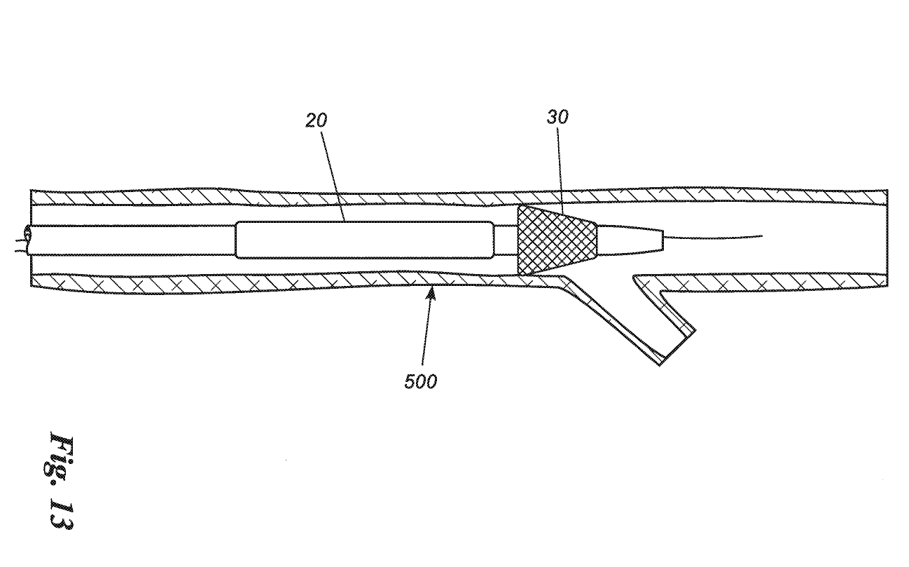

In FIG. 11 the embolic filter 30 has been expanded by pulling on the actuator wire 84. In FIG. 12 the angioplasty balloon 20 can be inflated and, if needed, deflated and re-inflated, optionally multiple times, to force the stenosis open. In the process of crushing the plaque that forms the stenosis, embolic particles 510 are released and swept by the blood flow into the open proximal end of the embolic filter 30, where they are captured by the filter membrane 50.

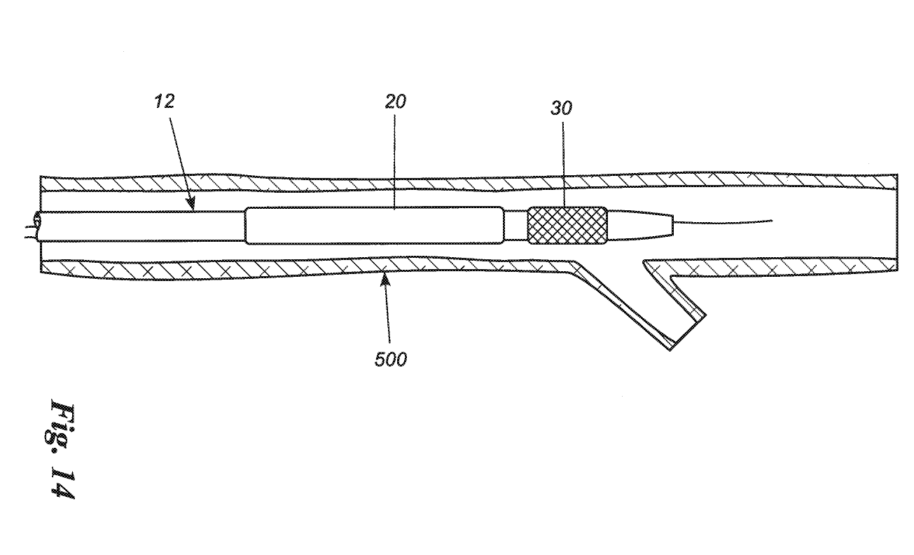

In FIG. 13, the formerly stenosed region can be open, and the angioplasty balloon 20 has been deflated. The embolic filter 30 remains open to capture any emboli released as the angioplasty balloon 20 deflates and pulls away from the wall of the vessel 500.

In FIG. 14, the embolic filter 30 can be closed, trapping captured emboli within the filter. The catheter 12 can now be withdrawn from the vessel 500.

One aspect of each of the disclosed embolic filters can be that, because the struts fold back on themselves, the filter scaffolding in its un-deployed position can be shorter than other known and/or commercially available embolic filters. The shorter length can enable a shorter landing zone, which in turn can permit the filter to be placed closer to the angioplasty treatment means. One result of providing a shorter landing zone can be a reduced likelihood that a branch blood vessel will intersect the stenosed blood vessel between the angioplasty treatment means and the embolic filter, thus reducing the chances of emboli bypassing the filter and getting caught up in the bloodstream.

Thus, implementations of the foregoing provide various desirable features. For instance, the present disclosure permits the placement of the embolic filter very close to the means for treating the stenosis. This has the effect of minimizing the “landing area” of the filter and also permits the protection of side branches, as shown in FIGS. 22-25.

The present invention can thus be embodied in other specific forms without departing from its spirit or essential characteristics. The described aspects are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. An apparatus comprising:
a catheter having an elongated shaft, proximal and distal ends, and a longitudinal axis; and
a filter membrane support structure, comprising:
a first ring coaxially fixedly mounted on a distal portion of said catheter shaft;
a second ring coaxially slideably mounted on a distal portion of said catheter shaft for movement toward and away from said first ring; and
a scaffolding extending between said first and second rings, said scaffolding comprising:
first longitudinal connecting members having a first end attached to said first ring and a second end extending toward said second ring;
second longitudinal connecting members having a first end attached to said second ring and a second end extending toward said first ring;
each of said first and second longitudinal connecting members having a bifurcation formed on said second end thereof, each of said bifurcations comprising first and second branches; and
means for connecting a branch on each of said first longitudinal connecting members to a branch on an opposite one of said second longitudinal connecting members.
